(19) United States
(12) Patent Application Publication
(10) Pub. No.: US 2024/0030661 A1
(43) Pub. Date: Jan. 25, 2024

(54) Method for manufacturing an assembly consisting of a plastic tube portion having at least one tube-wire insert and a terminal connector

(71) Applicants: REHAU Industries SE & Co. KG, Rehau (DE); RAUMEDIC AG, Münchberg (DE)

(72) Inventors: Konstantin BECKSTEIN, Rehau (DE); Marco DAMKE, Schönwald (DE); Ricardo EHRENPFORDT, Zwickau (DE); Constanze RANFELD, Rehau (DE); Thomas RUHLAND, Bayreuth (DE)

(73) Assignees: REHAU Industries SE & Co. KG, Rehau (DE); RAUMEDIC AG, Münchberg (DE)

(21) Appl. No.: 18/027,075

(22) PCT Filed: Sep. 28, 2021
(86) PCT No.: PCT/EP2021/076563

§ 371 Date: Mar. 17, 2023

(30) Foreign Application Priority Data
Oct. 1, 2020 (DE) 10 2020 212 441.8

(51) Int. Cl. H01R 13/66; H01R 43/20

(52) U.S. Cl. CPC H01R 13/665; H01R 43/20

(57) ABSTRACT

In order to manufacture an assembly consisting of a plastic tube portion having at least one tube-wire insert and a terminal connector, an electrical conducting component group is first prefabricated. Conducting components of the conducting component group provide an electrical connection with the at least one tube-wire insert, in the assembled state. A conducting component configuration is separated from the prefabricated conducting component group. The conducting component configuration is electrically contacted with the at least one tube-wire insert. The terminal connector is connected to the plastic tube portion. This results in an assembly manufacturing method which is suitable for mass production. The conducting component group is part of a multi-lead frame which has a plurality of different, selectable conducting component configurations that can be specified by selecting appropriate conducting components depending on the electrical contacting requirements.

### CROSS-REFERENCES TO RELATED APPLICATIONS

This application claims the priority of German Patent Application, Serial No. DE 10 2020 212 441.8, filed Oct. 1, 2020, the content of which is incorporated herein by reference in its entirety as if fully set forth herein.

### FIELD OF THE INVENTION

The invention relates to a method for manufacturing an assembly consisting of a plastic tube portion having at least one tube-wire insert and a terminal connector. Furthermore, the invention relates to an assembly manufactured by means of the method and to a multi-lead frame having a conducting component group for use within the method.

### BACKGROUND OF THE INVENTION

A plastic tube portion having a wire insert is known through obvious prior use. EP 0 142 944 A2 discloses means for self-locking electrical and mechanical connection in the context of a plastic tube portion in which helical wire inserts are incorporated. DE 11 2014 005 831 T5 discloses humidifi-cation system connections.

### SUMMARY OF THE INVENTION

It is an object of the present invention to provide a manufacturing method of the type mentioned above which is suitable for mass production.

This object is achieved according to the invention by a method for manufacturing an assembly consisting of a plastic tube portion having at least one tube-wire insert and a terminal connector, comprising the steps of prefabricating an electrical conducting component group, the conducting components of which, in the assembled state, provide an electrical connection with the at least one tube-wire insert, separating a conducting component configuration from the conducting component group, electrically contacting the conducting component configuration with the at least one tube-wire insert, connecting the terminal connector to the plastic tube portion.

According to the invention, it has been recognized that an electrical contact between the at least one tube-wire insert and either the terminal connector or a further component connected thereto can be simplified and, in particular, standardized by using a prefabricated electrical conducting component group. This further component may be a component integrated into the terminal connector and/or a component arranged separately from the terminal connector. The manufacturing method can be largely or completely automated. The terminal connector can be made of plastic. Electrical contacting of the conducting component configuration with the at least one tube-wire insert may be performed by soldering, by stirrup soldering, by welding, by bonding, by gluing or by crimping. By means of electrical contacting, an electrical as well as mechanical connection can be provided between the tube-wire insert and the conducting components of the conducting component configuration. A dome/mandrel can be inserted as a counter support during electrical contacting. It is particularly advantageous if such a dome or mandrel serves as a stop shape for bending the conducting components during assembly manufacture. The conducting components of the conducting component configuration may have contacting pads and/or plug members. The conducting components of the conducting component configuration may have additional contacting structures for electrically contacting further components independently of the terminal connector. A typical di-ameter of the conducting components of the conducting component configuration can be smaller than 0.5 mm and can be in the range between 50 μm and 200 μm.

The prefabricated electrical conducting component group may have conducting components and retaining components retaining the latter. The conducting components of the conducting component group are present within the conducting component group in at least one conducting component configuration. When separating a conducting component configuration to be selected from the conducting component group, retaining components and/or unneeded conducting components may be separated from the selected conducting component configuration.

Upon separation, the conducting component configuration is separated from at least one remaining component of the conducting component group.

The manufacturing method may include equipping the terminal connector with a component to be electrically and/or mechanically connected, in particular an electronic component and/or a sensor component, and/or covering the terminal connector.

The at least one terminal connector of the assembly can be arranged at one end of the plastic tube portion. Alternatively or additionally, the at least one terminal connector can also be connected to a tube sheath of the plastic tube portion at another position of the plastic tube portion between its ends. For the preparation of such a terminal connector, the plastic tube portion can be stripped in an assembly portion of the terminal connector in such a manner that the tube-wire insert is accessible from the outside at least in portions.

A signal transmission and/or a current or voltage supply can take place via the tube-wire insert. The wires of the wire insert can have a round cross-section or a cross-sectional shape that deviates from a round cross-section. The at least one wire of the wire insert may be a metallic wire and/or a plastic wire. In this case, the plastic wire includes, for example, at least one conductive polymer.

The assembly can be used in particular in connection with the use of smart sensors. For example, the assembly can be connected to an external control system and/or other components of a larger system.

The assembly can be used within the scope of the following applications:
heated tubes for hot drinks, for example coffee, in particular made of silicone;
brake lines/hydraulic tubes, in particular for bicycles;
cooling tubes, in particular for X-ray systems;
silicone tubes in general, in particular for white goods (refrigerators, washing machines, dishwashers . . . );
data lines in general.

Corresponding or additional applications of the assembly are, for example, heatable tubes, coolant tubes with wire insert, in particular for contacting integrated temperature sensors, hydraulic tubes with signal lines for monitoring hydraulic brakes. Other medical and/or automotive or other mechanical engineering applications of the assembly are also possible.

In the prefabrication and electrical contacting, wherein the conducting components of the prefabricated electrical conducting component group, in the assembled state, provide an electrical connection between the at least one tube-wire insert and associated electrical terminals of the electrical terminal connector, the terminal connector is electrically contacted with the at least one tube-wire insert via the conducting component configuration. Via this, an electrical connection with a component that is accommodated in the terminal connector, for example with a sensor module, can be established. Alternatively or additionally, it is possible to provide a connection with terminal contacts of the terminal connector via which the latter can in turn be electrically contacted, for example via a plug connection, with a further component. Alternatively, it is possible that the terminal connector itself does not have its own electrical terminal contacts, wherein an electrical connection with another component, in particular an electronic component, is then established via free ends of the conducting components of the conducting component configuration.

Prefabrication of the conducting component group and the separation of the conducting component configuration which is performed by producing a lead frame comprising the conducting component configuration and a frame carrier component, and by punching out the conducting component configuration from the lead frame has proven itself in practice. The lead frame can be prefabricated as a mass product. The lead frame can be a grid made of metal, in particular copper. Alternatively, the lead frame may also be a thermoset conduction carrier, for example made of PCB, and/or a conductor board.

After punching the conducting component configuration and prior to electrically contacting the latter to the at least one tube-wire insert, component portions of the conducting component configuration may be bent to increase a contact area between those components and the associated wires of the wire insert. Such a bending step may improve a definition of a relative positioning of the conducting component configuration on the tube-wire insert.

The conducting component configuration that is punched out of the lead frame can be the final conducting component configuration that is used in the manufacturing method. Alternatively, it is possible to punch out a conducting component configuration including surplus carrying components, wherein the surplus carrying components, in particular remaining webs, can then be subsequently separated from the conducting components of the conducting component configuration towards the end of manufacture, in particular via predetermined breaking points. Corresponding predetermined breaking points are not mandatory. The use of such a lead frame increases the robustness of the conducting components during electrical contacting as well as during connection of the terminal connector, in particular during overmolding of portions of the conducting components with plastic material of the terminal connector. If necessary, surplus carrying components of the conducting component configuration can be removed only after the connection of the terminal connector with plastic tube portion in order to keep the conducting components and their free ends serving a subsequent electrical contacting in a correct position during this connection step, in particular during overmolding. Upon separation, the conducting component configuration can be specified which is actually used later after electrical contacting in the assembly, in particular for signal transmission; however, a configuration can also be separated which, in addition to the actual conducting component configuration, also contains supporting or carrying members in order to ensure robustness of the conducting component configuration that is actually used for transmission during the electrical contacting and the connection of the terminal connector.

An extrusion method in which the plastic tube portion having the tube-wire insert is manufactured by extrusion has proven itself in practice. If the tube-wire inserts are made of plastic, this can be a coextrusion method.

A stripping step in which the at least one tube-wire insert is stripped before the electrical contacting enables safe electrical contacting of the at least one tube-wire insert.

A surface coating, at least in portions, of the at least one tube-wire insert which is carried out prior to the electrical contacting, can be used in particular by a low-resistance electrical contacting. Corrosion of contact regions of the tube-wire insert can also be avoided.

A surface of the lead frame can also be coated in the region, in particular, of contact pads of the conducting components and possibly of further contact points with the wire insert. The coating can be a tin coating.

A method comprising equipping the conducting component configuration and/or the terminal connector with an electronic component leads to an assembly which is ready for sensory use. For example, a pressure sensor and/or a temperature sensor or also a sensor for chemical analysis can be used as an electronic component or sensor component. Also, for example, a humidity sensor or an acceleration sensor can be used. A sensor for deter-mining a gas flow or an amount of liquid transported through the tube portion can also be used.

A mechanical connection comprising an additional mechanical connection of the plastic tube portion to the terminal connector results in a robust assembly. The mechanical connection of the terminal connector to the plastic tube portion can be achieved by overmolding the plastic tube portion and the conducting component configuration. The overmolding can in particular protect the conducting component configuration. Undesired corrosion and/or undesired short circuits can be avoided.

Alternatively, a mechanical connection between the terminal connector and the plastic tube portion can also be established exclusively via the electrical contact.

An injection molding manufacture of the terminal connector has proven itself in practice.

An injection molding step in which injection molding of the terminal connector takes place after electrical contacting of the conducting component configuration with the tube-wire insert enables the terminal connector to be produced in the course of assembly manufacture. A prefabricated assembly of the plastic tube portion and the conducting component configuration that is electrically contacted therethrough can then be used as an insert in an injection molding tool used to manufacture the terminal connector. The fluidic channel and the sensor surface can be kept free in the injection molding process by means of a dome or mandrel and, if necessary, further slides.

The advantages of an assembly manufactured by means of a method according to the above description, with a plastic tube portion having at least one tube-wire insert, with a terminal connector and with a conducting component configuration for electrical contacting with the tube-wire insert correspond to those already explained above with reference to the manufacturing method.

This applies accordingly to the assembly configured such that the tube-wire insert has a surface coating at least in sections, configured such that the conducting component configuration has contacting pads and/or connector elements and comprising at least one electronic component that is electrically contacted to the at least one tube-wire insert via the conducting component configuration and to a multi-lead frame having a conducting component group for use within a method according to the above description, comprising a plurality of different selectable conducting component configurations which can be specified by selecting respective conducting components according to electrical contacting requirements. The latter can be used flexibly for different assemblies to be manufactured, which differ in particular in their electrical contacting requirements.

The multi-lead frame may have a frame carrier component that carries the conducting component group. Components of the conducting component group that are not used for a respectively selected conducting component configuration can then be part of the frame carrier component. Such components that are not used for electrical transmission can also be punched out first and removed later and, if necessary, even after electrical contacting.

Examples of embodiments of the invention are explained below with reference to the drawings, in which:

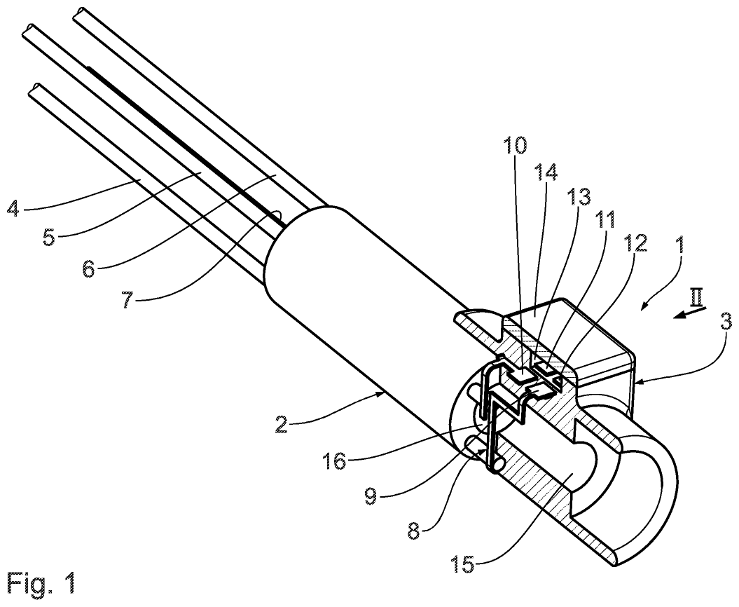

FIG. 1 in the region of a terminal connector, shows a longitudinally cut assembly consisting of a plastic tube portion including a total of four tube-wire inserts and a terminal connector that is contacted therewith at the end face via a conducting component configuration;

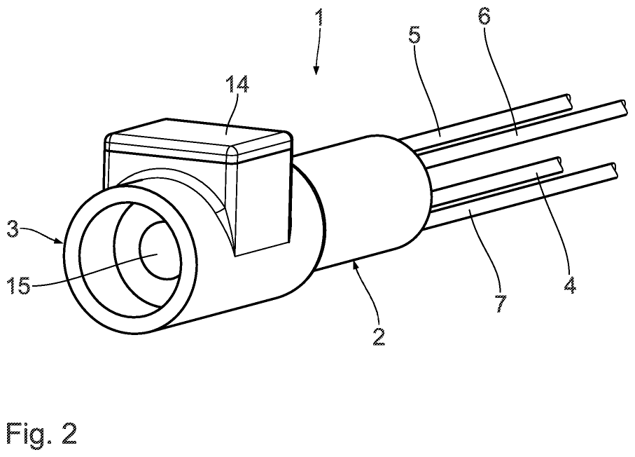

FIG. 2 shows a view onto the assembly from view direction II in FIG. 1;

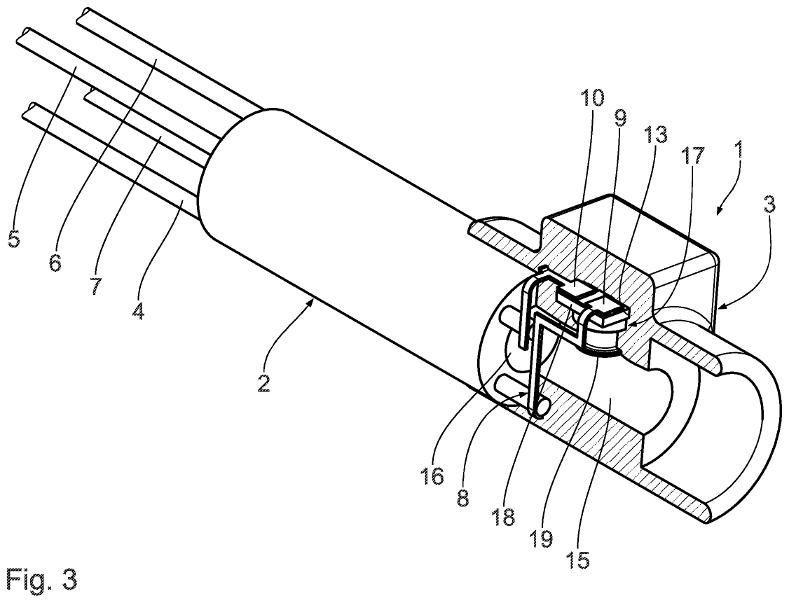

FIG. 3 shows a further embodiment of an assembly similar to FIG. 1, in which the embodiment of the terminal connector according to FIG. 1 is replaced by an embodiment of a terminal connector having a sensor component in the form of a sensor module;

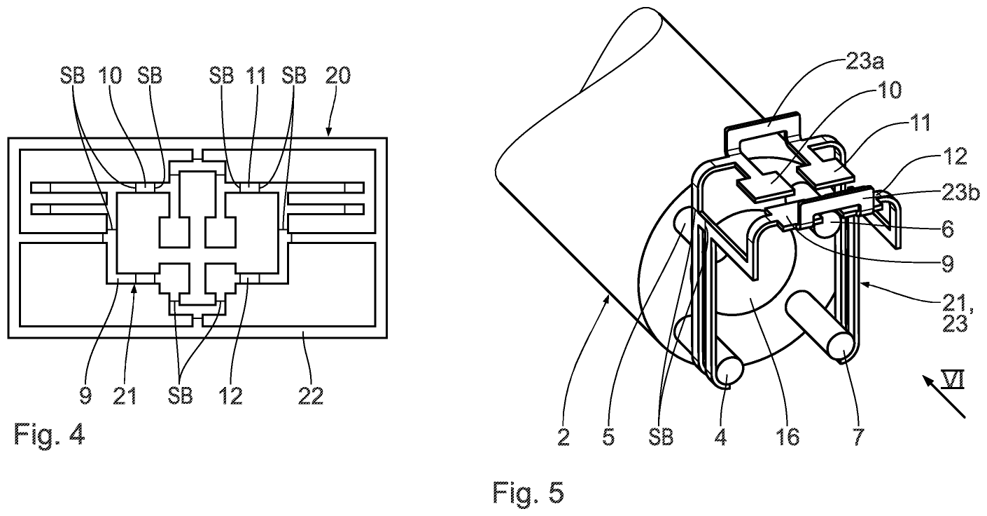

FIG. 4 shows a lead frame with a conducting component group from which a conducting component configuration corresponding to the conducting components according to FIGS. 1 and 3 can be selected by corresponding punching;

FIG. 5 in an illustration similar to FIGS. 1 and 3, shows the plastic tube portion with an embodiment of the conducting component configuration electrically which is contacted with all four tube-wire inserts such that any two of the four tubing wire inserts are electrically contacted with each other, using the entire electrical conducting component group of the lead frame shown in FIG. 4;

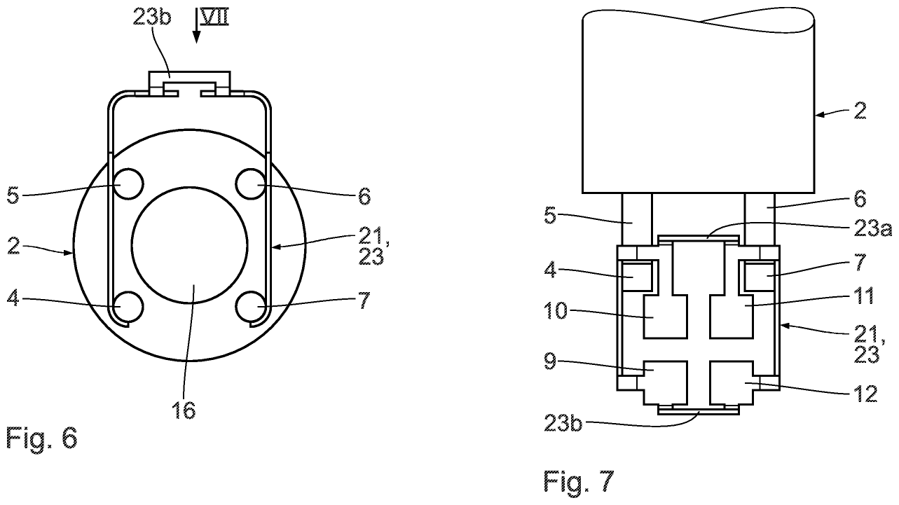

FIG. 6 shows a front view according to view direction VI in FIG. 5;

FIG. 7 shows a top view according to view direction VII in FIG. 6;

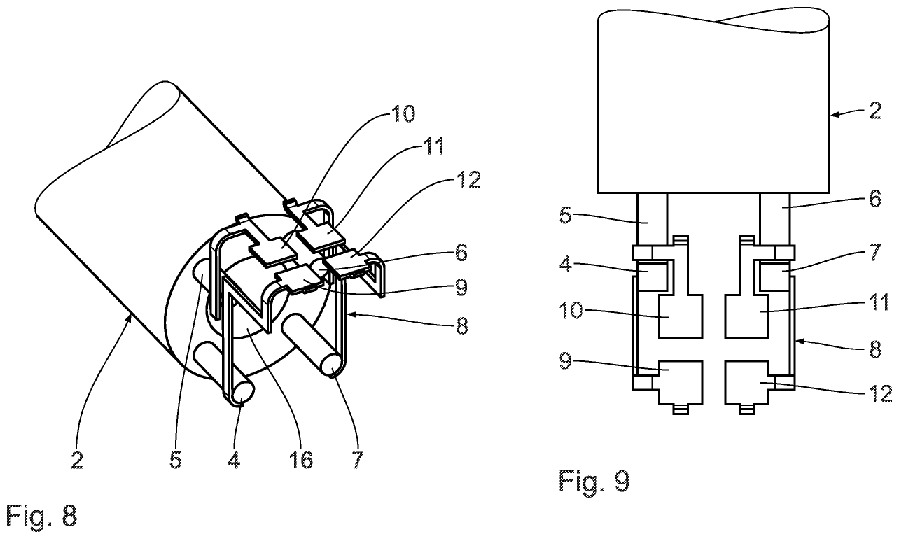

FIG. 8 shows an illustration similar to FIG. 5 of the tube portion with the tube-wire insert and a variant of the conducting component configuration, in which all four tube-wire inserts are contacted independently and insulated from one another with conducting components of the conducting component configuration, wherein the conducting component configuration according to FIG. 8 can emerge from the configuration according to FIG. 5 or from the arrangement in the lead frame according to FIG. 4 by separating corresponding predetermined breaking points;

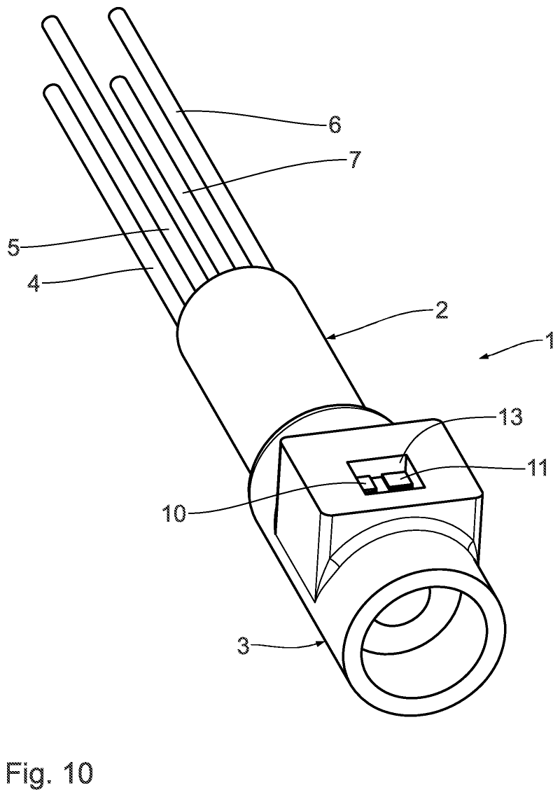

FIG. 9 shows a top view similar to FIG. 7 of the conducting component configuration according to FIG. 8;

FIG. 10 shows an embodiment of the assembly with the plastic tube portion and the conducting component configuration according to FIG. 8 and a terminal connector that is produced by overmolding in an injection molding process before contacting with additional components, in particular with sensor components, and before covering the terminal connector;

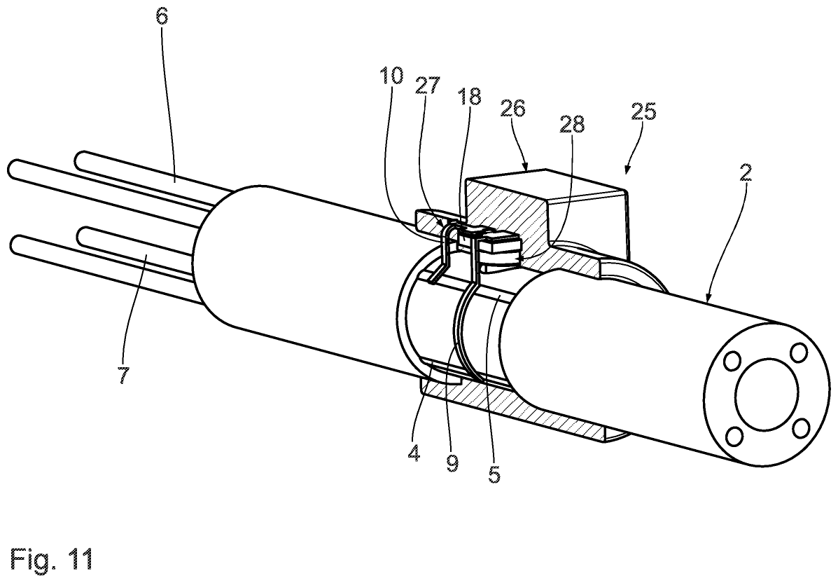

FIG. 11 shows a partially broken-down illustration of a further embodiment of an assembly consisting of a plastic tube portion with a total of four tube-wire inserts and a terminal connector contacted therewith on the sheath side via a conducting component configuration, as well as with a sensor component;

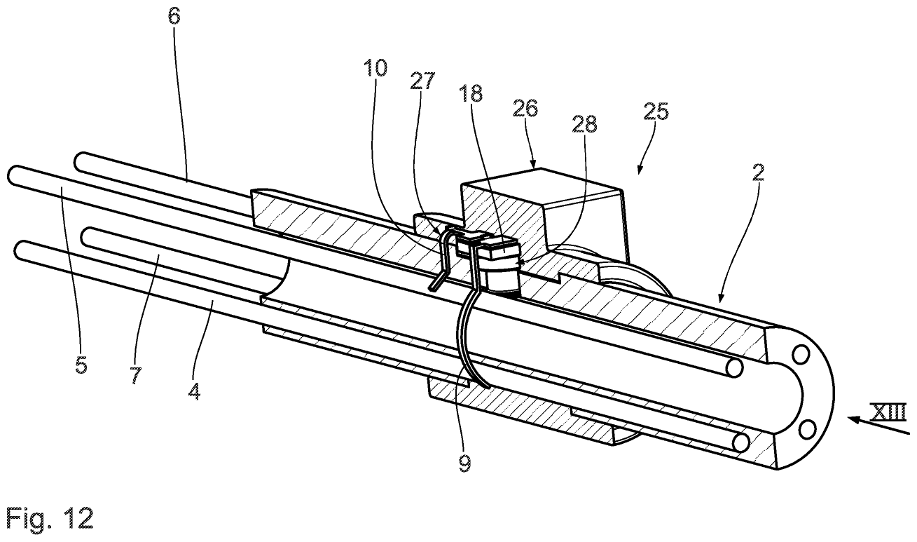

FIG. 12 shows a longitudinal section through the embodiment according to FIG. 11 in the region of the plastic tube portion;

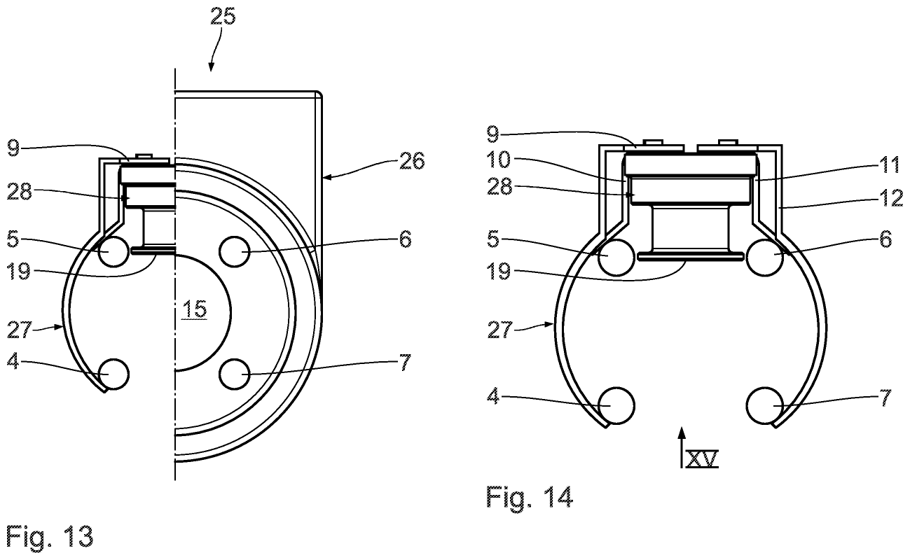

FIG. 13 shows a half-broken, front view onto the further embodiment, seen from view direction XIII in FIG. 12;

FIG. 14 shows the view according to FIG. 13 with the plastic tube portion omitted;

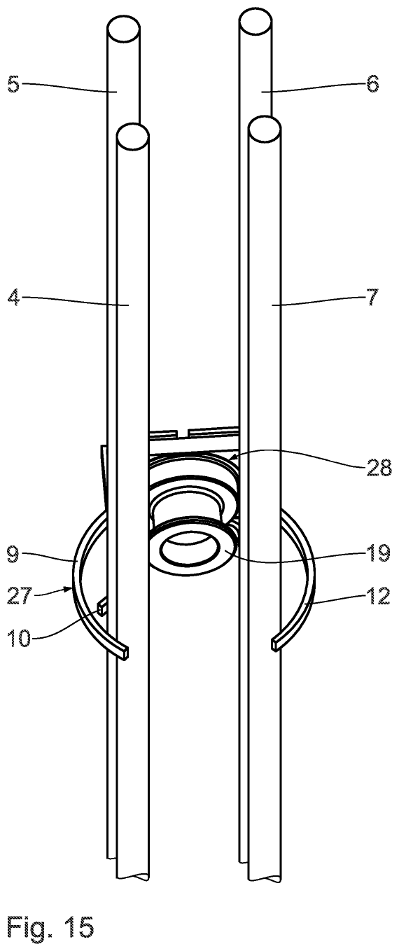

FIG. 15 shows a perspective bottom view approximately from view direction XV in FIG. 14; and

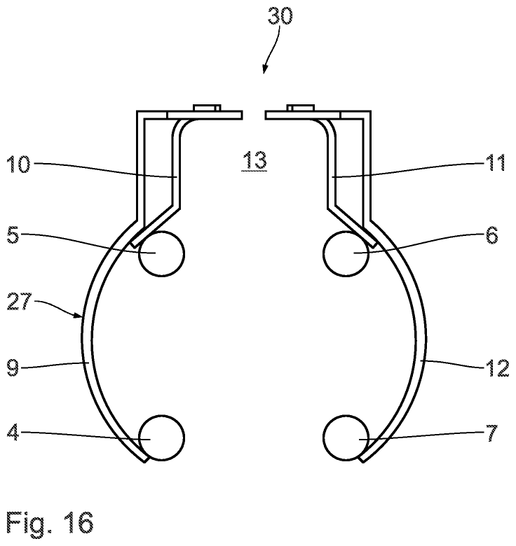

FIG. 16 in an illustration similar to FIG. 14, shows an embodiment of components of the assembly without a sensor component.

FIG. 1 shows an assembly 1 which can be used in a sensor application in connection with a fluid guide through a tube. The assembly can, for example, be part of a patient ventilation system. Applications of the assembly 1 are, for example, heatable tubes, coolant tubes with wire insert, in particular for contacting integrated temperature sensors, hydraulic tubes with signal lines for monitoring hydraulic brakes. Other medical and/or automotive or other mechanical engineering applications of assembly 1 are also possible.

The assembly 1 has a plastic tube portion 2 and a mechanical and/or electrical terminal connector 3. The tube portion has a total of four tube-wire inserts, i.e. four conductive wires 4, 5, 6, 7 (cf. also FIG. 2). The plastic tube portion is manufactured with the wire inserts 4 to 7 by extrusion.

The wires of the wire inserts 4 to 7 can be electrical conductor elements made of copper, aluminum, silver or electrically conductive polymers. Conducting elements with non-circular cross-sections, for example sheet metal and plastic conductive elements, are also to be understood as wires in the following. The wire inserts 4 to 7 protrude from the end face of the plastic tube portion 2. The wire inserts 4 to 7 can have a surface coating, for example of tin or silver, at least in portions.

The terminal connector 3 is made of plastic.

A conducting component configuration 8 with conducting components 9, 11 and 12 is used for the electrical contacting of the wire inserts 4 to 7 with the associated electrical terminals of the terminal connector 3. Only end contact pads of the conducting components 11 and 12 can be seen in FIG. 1. Instead of contact pads, plug elements can also be provided at the free end of the conducting components 9 to 12. The conducting component 9 is electrically connected to the wire 4. The conducting component 10 is electrically connected to the wire 5. The conducting component 11 is electrically connected to the wire 6. The conducting component 12 is electrically connected to the wire 7. The conducting components 9 to 12 are electrically contacted with the wires 4 to 7. This electrical contact can be established by soldering, stirrup soldering, welding, bonding, gluing or crimping. The conducting components 9 to 12 can have additional contacting structures for mechanical and electrical contacting of further, in particular electronic external components, in particular sensor components, electronic components and modules, micro-controllers, LEDs, further conductor boards, powerline communication chips and/or other components.

The routing of the conducting components 9 to 12 is such that these conducting components 9 to 12 are insulated from each other. The end-side contact pads of the conducting components 9 to 12 are arranged in an installation space 13 in which a further component, in particular a sensor component, can be accommodated in the terminal connector 3. A sensor of the sensor component can be a thermal sensor and/or a pressure sensor. Alternatively or additionally, it is possible to accommodate a signal-pro-cessing unit, for example a processor, in the installation space 13 via the conducting components 9 to 12. In particular, an assembled printed circuit board can be accommodated in the installation space 13. Via the wires 4 to 7 and the conducting components 9 to 12 of the conducting component configuration 8, signals can be transmitted from and to the component of the terminal connector 3 that is to be accommodated in the installation space 13.

Depending on the requirements, the installation space 13 is used for electrical contacting of an external component, which is also not shown, by means of a cable or plug that is not shown.

In particular, fluidic sensor components can be oriented towards the lumens 15, 16 in the installation space 13. Electronic components that do not re-quire media access to the medium guided in the lumen 15, 16 can also be overmolded when overmolding of the terminal connector 3 takes place, so that no separate installation space needs to be provided for these components.

The installation space 13 is covered with a lid 14. A plug for contacting the conducting components 9 to 12 with an external component can be arranged in the lid 14. Such a plug can alternatively or additionally be overmolded with the terminal connector 3.

The terminal connector 3 has a connector lumen 15 which is aligned with a tube lumen 16 of the tube portion 2. A region of the tube portion 2 facing the terminal connector 3 can, together with the conducting component configuration 8, form an insert for an injection molding tool with which the terminal connector 3 is manufactured still without the lid by overmolding this insert.

The terminal connector 3 can be designed as a carrier for a sensor module 17. Alternatively, the conducting component configuration 8 can serve as a carrier for the sensor module 17. The sensor module 17 can be electrically contacted with the contact pads of the conducting components 9 to 12 and later be overmolded when connecting the terminal connector. If media ac-cesses or sensitive sensor structures are not to be overmolded, they can be protected from overmolding by sliders during the injection molding process.

In the embodiment according to FIG. 3, a conductor board 18 contacts the sensor module 17 on the one hand with the conducting components 9 to 12 on the other hand. The sensor module 17 has a sensor surface 19 oriented towards the connector lumen 15. The sensor surface 19 can be a pressure- and/or temperature-sensitive sensor surface. A sensor surface that allows the detection of a concentration of a medium or a proportion of a certain composition of a medium that is guided via the lumens 15, 16 can also be used.

FIG. 4 shows a multi-lead frame 20 with a conducting component group 21, from which a conducting component configuration in the manner of the conducting component configuration 8 can be selected and separated for manufacturing the assembly 1 according to FIG. 1 or 3. The conducting components 9, 10, 11, 12 are components of the conducting component group 21 and are connected to each other and to an outer circumferential frame carrier component 22 of the multi-lead frame 20 via various predetermined breaking points SB.

The multi-lead frame 20 can be provided in rolls.

The multi-lead frame 20 contains a plurality of different, selectable conducting component configurations, which can be selected according to the requirements for electrical contacting by separating the various predetermined breaking points of the conducting component group 21. Components of the conducting component group 21 of the multi-lead frame 20 that are not used for the respective selected conducting component configuration, i.e. for example the conducting component configuration 8 according to FIGS. 1 and 3, can then serve as part of the frame carrier component.

FIG. 5, in a representation similar to FIGS. 1 and 3, shows the plastic tube portion 2 with the wire inserts 4 to 7 and a variant of a conducting component configuration 23 which can be used instead of the conducting component configuration 8 and which at the same time represents the complete conducting component group 21 of the multi-lead frame 20. Retaining bridges of the conducting component configuration 23, which are still to be removed after the electrical contacting of this configuration 23 with the wires 4 to 7 of the wire insert, are marked 23a and 23b in FIG. 5. These retaining bridges increase stability of the initially separated conducting component configuration 23. Contacting of the wire inserts 4 and 5 on the one hand and 6 and 7 on the other hand is provided via this conducting component configuration 23. Signals which are given via the contact pads to the free ends of the conducting components 9 to 12 of the conducting component configuration 23 are present simultaneously on the one hand on the wires 4 and 5 and on the other hand simultaneously on the wires 6 and 7 of the tube portion 2.

After attaching the conducting component configuration 23 to the free ends of the wire inserts 4 to 7 and before making final electrical contact with the latter, the conducting component configuration 8 according to FIGS. 1 and 3 can be created by separating corresponding portions of the conducting component group 21 (cf. predetermined break points SB, indicated in FIGS. 4 and 5), as is also made clear by comparing FIG. 5, which shows the conducting component configuration 23, with FIG. 8, which shows the conducting component configuration 8.

FIG. 10 shows the assembly 1 according to FIG. 2 before it is covered, before it is contacted with a plug or cable and before the installation space 13 of the terminal connector 3 is equipped with further electronic components.

When the assembly 1 is manufactured, the conducting component group 21 is first prefabricated in the form of the multi-lead frame 20. Then the de-sired conducting component configuration, for example the conducting component configuration 8, is in each case selected from the conducting component group 21 and separated. This is done by punching out and, if necessary, subsequently separating the conducting components 9 to 12 from the conducting component group 21 of the multi-lead frame 20.

Thereafter, the conducting component configuration is electrically contacted with at least one of the wires 4 to 7, i.e. with all wires 4 to 7 if the conducting component configuration 8 is used. In this process, partial portions of the conducting components 9 to 12 that are associated with the wires 4 to 7 are bent over accordingly so that a contact area between these partial portions of the conducting components 9 to 12 and the facing lateral surfaces of the wires 4 to 7 is enlarged. Subsequently, soldering, in particular stirrup soldering, welding, bonding, gluing or crimping is carried out to establish the electrical contact.

Via the free ends of the conducting components 9 to 12, electrical contacting can then take place with a component that is accommodated in particular in the terminal connector 3, for example with a sensor component in the form of the sensor module 17, which again can be performed by one of the contacting methods. If the conducting components 9 to 12 are provided with plug elements instead of the contacting pads, this contacting can also be carried out by plugging.

Prior to electrical contacting, the insulation of the at least one tube-wire insert 4 to 7 can be stripped, in particular by removing a region of the plastic tube portion.

As part of the manufacturing method, the terminal connector 3 is connected to the plastic tube portion 2. This can be done by overmolding the electrically contacted plastic tube portion including the conducting component configuration with the plastic material of the terminal connector 3. Due to the overmolding, the terminal connector 3 is mechanically connected to the plastic tube portion 2. After overmolding, electronic components, in particular sensor components, can be inserted into the installation space 13 of the terminal connector 3. The electronic component can be aligned with the lumen 15, 16 of the assembly 1, as shown for example in FIG. 3. Additional circuit boards, controllers and further electrical components beyond a sensor component, for example, can also be integrated into the installation space 13.

With reference to FIGS. 11 to 15, a further embodiment of an assembly 25 is described below, which can be used instead of or in addition to assembly 1. Components and functions that correspond to those already explained above with reference to FIGS. 1 to 10 bear the same designations and, where applicable, reference signs and are not discussed again in detail.

In the assembly 25, a terminal connector 26, which otherwise corresponds to the terminal connector 3, is not attached at the end face to one end of the plastic tube portion 2, but on the sheath side, i.e. in the region of a tube sheath of the plastic tube portion 2. For this purpose, the tube sheath of the plastic tube portion 2 is stripped in the region of a mounting portion of the terminal connector 26, so that the wires 4 to 7 are exposed to the outside. Conducting components 9 to 13 of a conducting component configuration 27, the function of which corresponds to that explained above in connection with conducting component configurations 8 and 23, are connected to the wires 4 to 7 of the tube-wire insert. Contact pads of the conducting components 9 to 12 of this conducting component configuration 27 are in contact with associated contacts of a sensor module 28 of the assembly 25, which in turn corresponds to the sensor module 17 with regard to its function.

The stripped region of the tube sheath, the conducting component configuration 27 and the sensor module 28 are again overmolded by the terminal connector 26 during the manufacture of the assembly 25. This overmolding produces in the manufacture of the assembly 25 in accordance with what has been explained above in relation to the assembly 1 according to FIGS. 1 to 10. During the overmolding process, plastic material also flows into gaps between the conducting components 9 to 12 and the wires 4 to 7, in order to avoid undesirable electrical contacts there. Corresponding insulation gaps between, for example, the conducting component 9 and the wire 5 that is not to be contacted with the latter are apparent from the end side views, for example, according to FIGS. 13 and 14.

The sensor module 28 again has a sensor surface 19 aligned with the connector lumen 15. This sensor surface 19 may be arranged to be recessed in the sensor module 28, as can be seen from the perspective illustration shown in FIG. 15.

In another embodiment of an assembly 30, in which only the wires 4 to 7 of the wire insert and the conducting component configuration 27 are shown in FIG. 16, the sensor module 28 is missing. In this case, the terminal connector 26 is overmolded around the plastic tube portion 2 in the region of the tube sheath of the stripped plastic tube portion 2 in such a manner that the installation space 13 remains accessible via a lid of the terminal connector 26. In this case, the assembly 30 can still be subsequently sup-plemented or completed by appropriate installation and/or contacting steps.

1. A method for manufacturing an assembly (1; 25; 30) consisting of a plastic tube portion (2) having at least one tube-wire insert (4 to 7) and a terminal connector (3; 26), comprising the following steps:
prefabricating an electrical conducting component group (21), conducting components (9 to 12) of which, in an assembled state, provide an electrical connection with the at least one tube-wire insert (4 to 7),
separating a conducting component configuration (8; 23; 27) from the conducting component group (21),
electrically contacting the conducting component configuration (8; 23; 27) with the at least one tube-wire insert (4 to 7),
connecting the terminal connector (3; 26) to the plastic tube portion (2).

2. The method according to claim 1, wherein the conducting components (9 to 12) of the prefabricated electrical conducting component group (21), in the assembled state, provide an electrical connection between the at least one tube-wire insert (4 to 7) and associated electrical terminals of the terminal connector, wherein during electrical contacting of the conducting component configuration (8; 23; 27) contacting takes place with the at least one tube-wire insert (4 to 7) and with the associated terminal of the terminal connector (3; 26).

3. The method according to claim 1, wherein the prefabrication of the conducting component group and the separation of the conducting component configuration (8; 23; 27) is performed by
producing a lead frame (20) comprising the conducting component configuration (8; 23; 27) and a frame carrier component (22), and
by punching out the conducting component configuration (8; 23; 27) from the lead frame (20).

4. The method according to claim 1, wherein the plastic tube portion (2) having the tube-wire insert (4 to 7) is manufactured by extrusion.

5. The method according to claim 1, wherein the at least one tube-wire insert (4 to 7) is stripped before the electrical contacting.

6. The method according to claim 1, wherein, prior to the electrical contacting, a surface coating, at least in portions, of the at least one tube-wire insert (4 to 7) is carried out.

7. The method according to claim 1, comprising equipping at least one of the conducting component configuration (8; 23; 27) and the terminal connector (3; 26) with an electronic component (17; 28).

8. The method according to claim 1, comprising an additional mechanical connection of the plastic tube portion (2) to the terminal connector (3; 26).

9. The method according to claim 1, wherein the terminal connector (3; 26) is manufactured as an injection molded part.

10. The method according to claim 9, wherein injection molding of the terminal connector (3; 26) takes place after electrical contacting of the conducting component configuration (8; 23; 27) with the tube-wire insert (4 to 7).

11. An assembly (1; 25; 30) manufactured by means of a method for manufacturing an assembly,
with a plastic tube portion (2) having at least one tube-wire insert (4 to 7),
with a terminal connector (3; 26) and
with a conducting component configuration (8; 23; 27) for electrical contacting with the tube-wire insert (4 to 7).

12. The assembly according to claim 11, wherein the tube-wire insert (4 to 7) has a surface coating at least in sections.

13. The assembly according to claim 11, wherein the conducting component configuration has contacting pads and/or connector elements.

14. The assembly according to claim 11, comprising at least one electronic component that is electrically contacted to the at least one tube-wire insert (4 to 7) via the conducting component configuration (8; 23; 27).

15. A multi-lead frame (20) having a conducting component group (21) for use within a method for manufacturing an assembly, comprising a plurality of different selectable conducting component configurations (8, 23; 27) which can be specified by selecting respective conducting components (9 to 12) according to electrical contacting requirements.
